(19) United States
(12) Patent Application Publication
(10) Pub. No.: US 2024/0075005 A1
(43) Pub. Date: Mar. 7, 2024

(54) Treatment of Overactive Bladder with Oxybutynin Applied By Means of a Vaginal Ring

(71) Applicant: LIGALLI BV, Den Haag (NL)

(72) Inventor: Wilhelmus Nicolaas Gerardus Maria De Laat, Den Haag (NL)

(21) Appl. No.: 18/274,646

(22) PCT Filed: Feb. 28, 2022
(86) PCT No.: PCT/EP2022/055003

§ 371 Date: Jul. 27, 2023

(30) Foreign Application Priority Data
Feb. 28, 2021 (EP) 21159807.3

(51) Int. Cl. A61K 31/216; A61K 9/00

(52) U.S. Cl. CPC A61K 31/216; A61K 9/0036

(57) ABSTRACT

The present invention relates to oxybutynin for use in the treatment of a medical condition, wherein oxybutynin is administered to the subject in need of treatment by means of a vaginal ring, that comprises a drug reservoir for the liquid formulation of oxybutynin and a pump for dispensing the oxybutynin. The medical condition comprises overactive bladder (OAB) or post-menopausal or aromatase inhibitor-induced hot flashes. The ring may further comprise a miniature electronic circuit board that controls the ring, and optionally a battery. The vaginal ring may be wirelessly connected to an external device for programming drug delivery or for transmitting and/or receiving data from the ring.

The present invention relates to a new type of administration of oxybutynin for the treatment of medical conditions, in particular overactive bladder and post-menopausal or aromatase inhibitor-induced hot flashes.

Overactive bladder (OAB) is a condition where there is a frequent feeling of need to urinate that can result in episodes of incontinence and anyhow negatively affects a person's life. The frequent need to urinate may occur during the day, at night, or both. Overactive bladder is prevalent in 16% of the population. It can for example be treated by medications that relax the bladder thus relieving the symptoms and reducing episodes of urge incontinence. These drugs include oxybutynin, which can be taken as a pill or used as a skin patch or gel. Such medications may lead to a high number of side-effects. In addition, many discontinuations occur due to the current forms of pharmacotherapy.

Aromatase inhibitors (AIs) are a class of drugs used in the treatment of breast cancer in postmenopausal women and in men, and gynecomastia in men. They may also be used to reduce estrogen conversion when supplementing testosterone exogenously and for chemoprevention in women at high risk for breast cancer. One of the known side-effects comprises hot flashes. Hot flashes can also occur in post-menopausal women. Oxybutynin is known to reduce the frequency and intensity of hot flashes. Many breast cancers have estrogen-positive receptors and thus the normal estrogen-containing hot flash medications are contra-indicated. Oxybutynin as a medication for obviating the side effects of AIs is a new method of non-hormonal medication for this indication.

Oxybutynin has an explicit place in the treatment of OAB and has been available on the market since 1975. Oxybutynin is a competitive acetylcholine antagonist for the postganglionic muscarine receptors. It results in a spasmolytic effect on the detrusor muscle in patients suffering from urge incontinence or pollakisuria (increased frequency of urination).

Systemic delivery of drugs is necessary to achieve therapeutic effects in various diseases and conditions. Systemic delivery of oxybutynin is usually obtained by oral or transdermal administration. Orally administered oxybutynin results in many side effects, including dry eyes and dry mouth, which are partially due to its metabolite, which is generated during hepatic first-pass metabolism. This extensive first pass effect results in the formation of the active metabolite N-desethyloxybutynin with systemic, anticholinergic side effects.

Treatment with transdermal patches, although circumventing first-pass metabolism, results in frequent unacceptable dermal side-effects, precluding extensive use.

It is the object of the present invention to provide an alternative and improved mode of administration of oxybutynin for the treatment of medical conditions such as OAB and hot flashes.

In the research leading to the invention, a vaginal ring was used as an alternative mode of administration. It was demonstrated that intravaginal administration of oxybutynin using a vaginal ring leads to observed plasma levels of oxybutynin. The plasma concentrations of oxybutynin and its metabolite N-desethyloxybutynin were well within the known safety window from the registered use of oxybutynin.

The parent:metabolite ratio was 1:1.3, which was well below the ratio of oral administration (>1:8), and in a similar range as the transdermal route with gel or patches, another route that bypasses the first-pass effect. This demonstrates that sufficiently high levels can be obtained while avoiding hepatic first-pass metabolism and corresponding side-effects.

The present invention thus relates to oxybutynin for use in the treatment of overactive bladder, wherein oxybutynin is administered to the subject in need of treatment by means of a vaginal ring, that comprises a drug reservoir for the liquid formulation of oxybutynin and a pump for dispensing the oxybutynin.

The present invention in another embodiment relates to oxybutynin for use in the treatment of hot flashes, wherein oxybutynin is administered to the subject in need of treatment by means of a vaginal ring. The ring used for this indication suitably comprises a drug reservoir for the liquid formulation of oxybutynin and a pump for dispensing the oxybutynin.

The pump provided in the vaginal ring used for administration according to the invention preferably comprises a miniature peristaltic pump The ring may further comprise a miniature electronic circuit board that controls the ring, and a battery. The vaginal ring is preferably wirelessly connected to an external device (smartphone, tablet, or laptop computer) from which drug delivery can be programmed and which transmits data to or receives data from the ring. The data received by the external device comprises for example volume delivered, temperature or any other parameter relevant to the treatment of OAB or hot flashes. The data transmitted by the external device comprises for example volume to be delivered, point of time of delivery, delivery pattern or any other parameter relevant to the treatment of OAB or hot flashes. A suitable embodiment of the vaginal ring is described in EP3359099 and is further referred to herein as the MedRing.

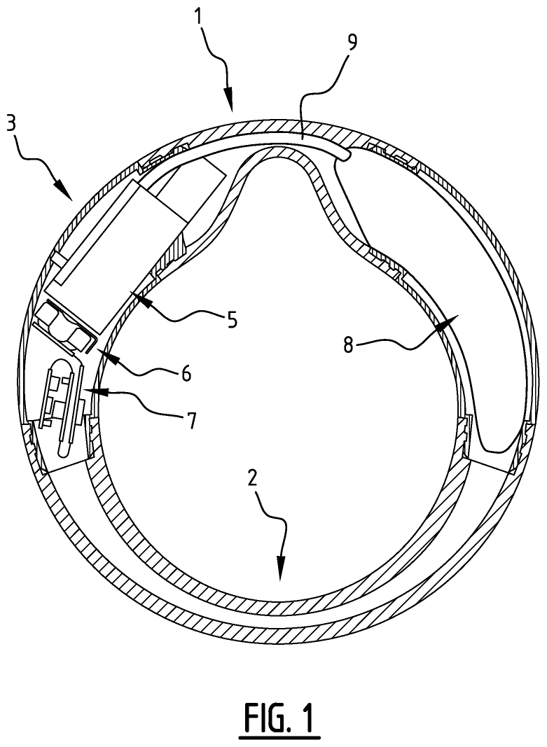

The MedRing is a flexible vaginal ring with a smooth surface (see FIG. 1 for a schematic cross-section of the ring). The MedRing contains a flexible segment for comfort and to allow intra-vaginal placement. The reservoir and the electronics-containing parts of the rings are contained in rigid enclosures.

The intra-vaginal delivery route offers several advantages for systemic drug delivery in general. It is a non-invasive route of administration, and it features a suitable residence time for long-term treatment.

An intravaginal route of administration of oxybutynin has advantages compared to oral administration. By this route it bypasses the first-pass effect. The proximity of the ring to the musculature of the bottom of the bladder, where the receptors for the antagonist are located, allows for a mechanism of “local effect”, i.e. the direct occupation of the receptors without the necessity of entering in the systemic blood circulation first. Moreover, the existence of the so-called “counter-current effect” of the special anatomy of genital blood circulation in the vagina, uterus, bladder and ovaries, that has proven to increase target level well above the blood levels, might leverage this effect further, allowing lower dosage and leading to better efficacy to side effect ratios. It has also advantages over transdermal administration since it does not affect the skin.

Furthermore, the MedRing is able to administer the compound according to various regimes, including pulsatile, on-demand, programmable dosing and time of administration or continuous drug delivery. The possibility of non-continuous administration by the novel mode of administration of the invention, as opposed to all other rings with continuous administration, will not exhaust the receptors and will lead to better efficacy.

The ring may further include a timer function, which allows further personalization of the treatment. For example, if the complaints only arise in the evening, the treatment can be given only in the evening.

The use of a vaginal ring for treatment of a medical condition facilitates treatment compliance since the ring may deliver the drug in an automated manner without interference of the patient.

It is also possible that if the absorption in the body is sufficiently fast, oxybutynin can be administered preventively before the complaints arise when the subject can feel the complaint coming.

In one embodiment, oxybutynin is administered by the ring in liquid formulation.

Oxybutynin can be administered by the ring in microliter dose that still leads to a sufficient systemic level.

In one embodiment, oxybutynin is administered by the ring at a single dose of 1-5 mg, preferably 3 mg. Oxybutynin is suitable used in a concentration of 50 to 200 mg/mL, preferably 75-150 mg/mL, more preferably about 100 mg/mL. These concentrations allow administration of microliter doses.

In one embodiment, the ring further comprises one or more diagnostic sensors.

Vaginal rings are known in the art. These rings are, however, limited by only being able to deliver a fixed dose, released continuously, and almost exclusively for restricted number of (gynecological) indications. These rings can neither be operated via an app, nor confirm drug compliancy, nor collect data.

In stark contrast with all existing vaginal rings, the present invention is based on the use of a novel and uniquely different vaginal ring. The ring contains miniaturized elements like a drug container, a pump, a battery, a motor, an antenna, electronics, and sensor(s) as shown in FIG. 1. Connectivity with a mobile device/telephone allows for adjustment of dose, schedule, and timing. The ring is discrete, elegant, invisible, and convenient to use. Further details about the ring can be found in EP3359099.

The ring allows adaptation of dose, schedule, and timing, via remote control with the patient's own smart phone. Furthermore, the ring collects and communicates physiological data. The ring is self-insertable and self-removable and is intended to function continuously for 4 weeks.

Furthermore, in one embodiment, the ring that is used in the invention is equipped with a temperature sensor, recording body temperature while being intravaginally in operation. This way, by reading in the log of the ring that body temperature was measured it can be ascertained that the ring has functioned properly. So, for the first time ever, adherence to the (non-invasive) administration of the prescribed medication is being monitored and confirmed by body temperature recordings of this sensor.

If the temperature sensor can predict the occurrence of a hot flash, the ring can provide a signal to the user allowing her to deliver oxybutynin on demand or the ring can give feed back to the pump to deliver oxybutynin in response to the signal.

Moreover, the medical doctor can accurately change the schedule and the dose based on the response of the patient to the treatment. This way, the ring allows better personalization and customization of the treatment of the patient.

According to the invention, it is thus demonstrated that it is possible to deliver oxybutynin to the human body with a vaginal ring that is provided with a drug release reservoir and a pump and obtain ratios between oxybutynin and its metabolite N-desethyloxybutynin that are lower than when oxybutynin is administered orally, thus leading to less metabolite-induced side effects. The MedRing as described herein is a suitable type of vaginal ring for delivering oxybutynin to the vagina.

Since it is known that the levels of oxybutynin that are now obtained by vaginal delivery of oxybutynin with the ring as described herein, i.e. by means of dispensing oxybutynin, preferably in liquid formulation, by a pump from a reservoir in a vaginal ring, are sufficient to have a therapeutic effect, it follows that this mode of administration is suitable for treatment of OAB and hot flashes.

The MedRing for use in the invention is illustrated in the cross-sectional view shown in FIG. 1. The vaginal ring comprises two flexible elements 1 and 2 and two rigid elements 3 and 4. Rigid element 3 comprises the pump 5, a battery 6 and the electronic components 7 necessary for controlling the pump and the wireless transceiver. Rigid element 4 is provided with a drug reservoir 8. The oxybutynin is transported from the reservoir 8 to the pump 5 through a tube 9.

The flexible parts are at least partially elastic, wherein the elasticity is such that the ring can be squeezed to transform a shape of the ring from an extended shape to a collapsed shape for allowing the ring to be inserted into a vagina of a user. The ring is pre-biased to assume the extended shape when little to no external force is being applied thereto, said extended shape corresponding to a substantially oval or annular ring shape. The ring assumes a shape substantially corresponding to the extended shape when the ring is inserted into the vagina with the squeezed rigid parts first. The rigid parts will assume a natural position in the deepest part of the vagina, the fornix posterior vaginae.

The present invention will be illustrated in the following example which is for illustration purposes only and is not intended to limit the invention in any way.

In the examples reference is made to the following figures:

FIG. 1: Schematic cross-sectional view of the MedRing for use in the invention.

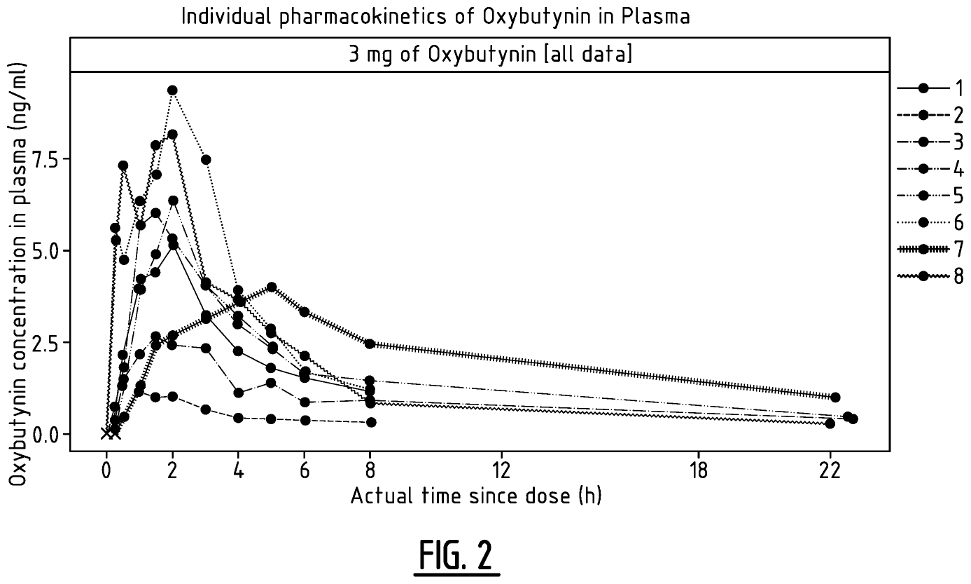

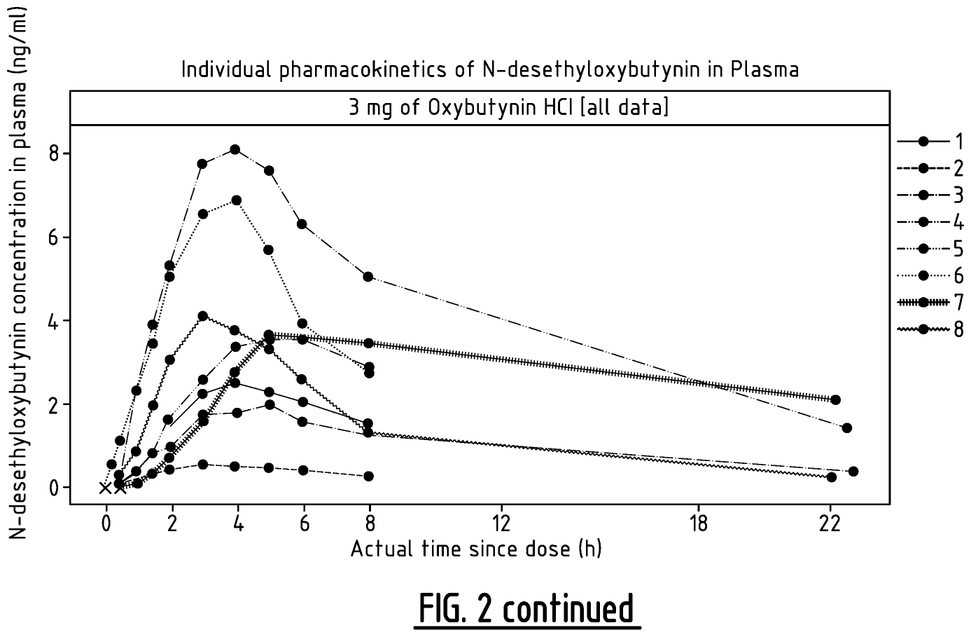

FIG. 2: Individual pharmacokinetic profiles of oxybutynin (upper) and N-desethyloxybutynin (lower) in plasma (ng/mL) following a single 3 mg intravaginal dose. The MedRing remained in situ for 2 hours in subject 1, 2, 5 and 6 and remained in situ for 6 hours in subject 3, 4, 7 and 8. Subjects 1-4 are pre-menopausal. Subjects 5-8 are post-menopausal.

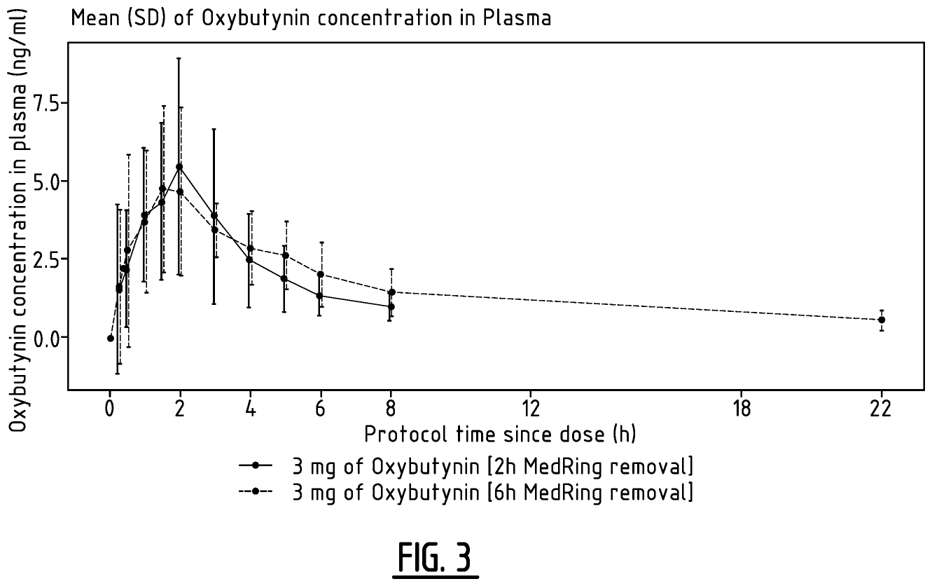

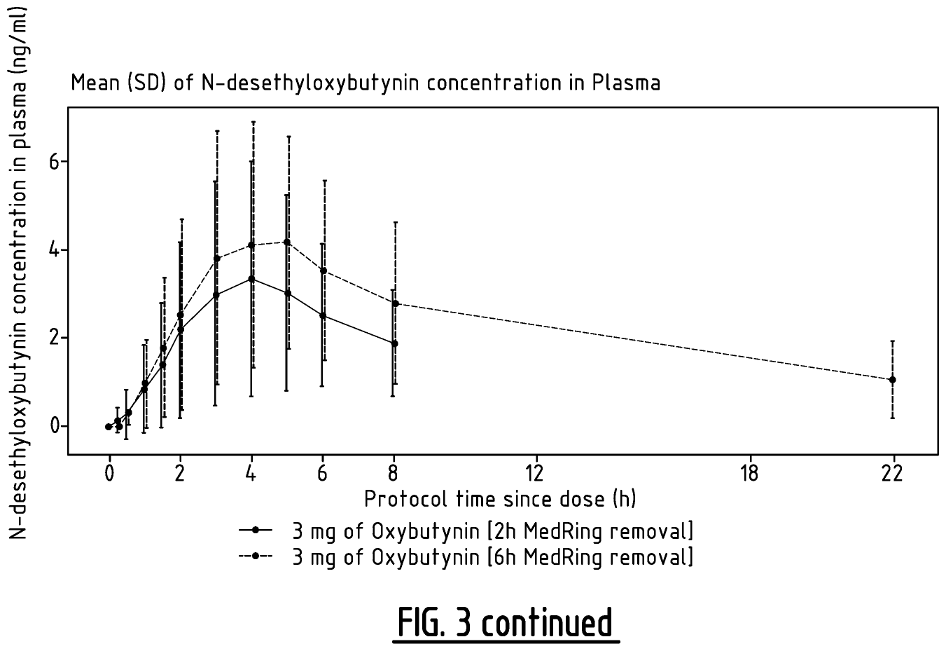

FIG. 3: Summary oxybutynin (upper) and N-desethyloxybutynin (lower) concentration in plasma (ng/mL). Solid line: the mean±standard deviation of oxybutynin concentration of the first cohort of four (4) subjects receiving 3 mg oxybutynin HCL are shown, in which the MedRing was removed after 2 hours. Dashed line: this is shown in the four (4) subjects of the second cohort, in which the MedRing was removed after 6 hours.

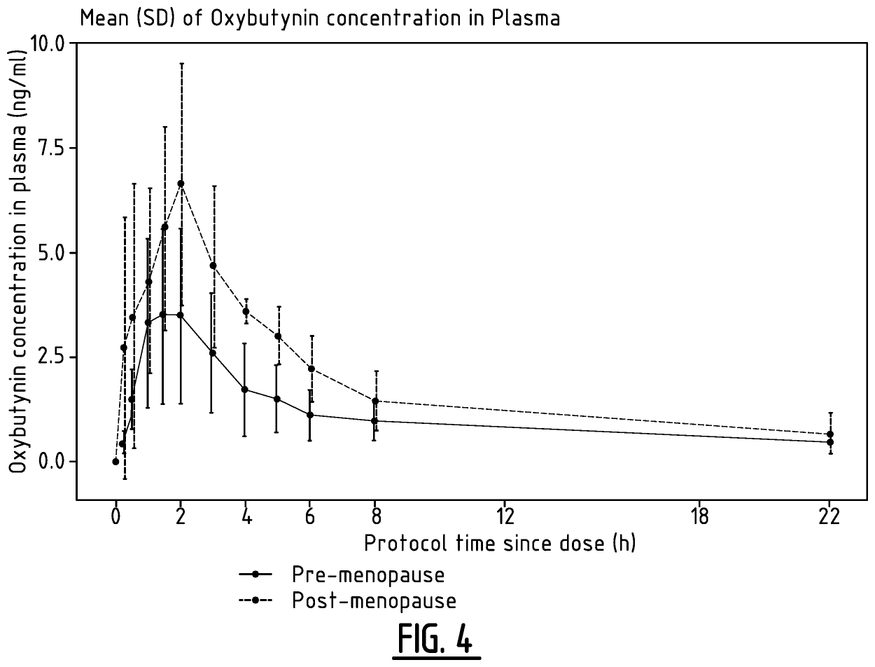

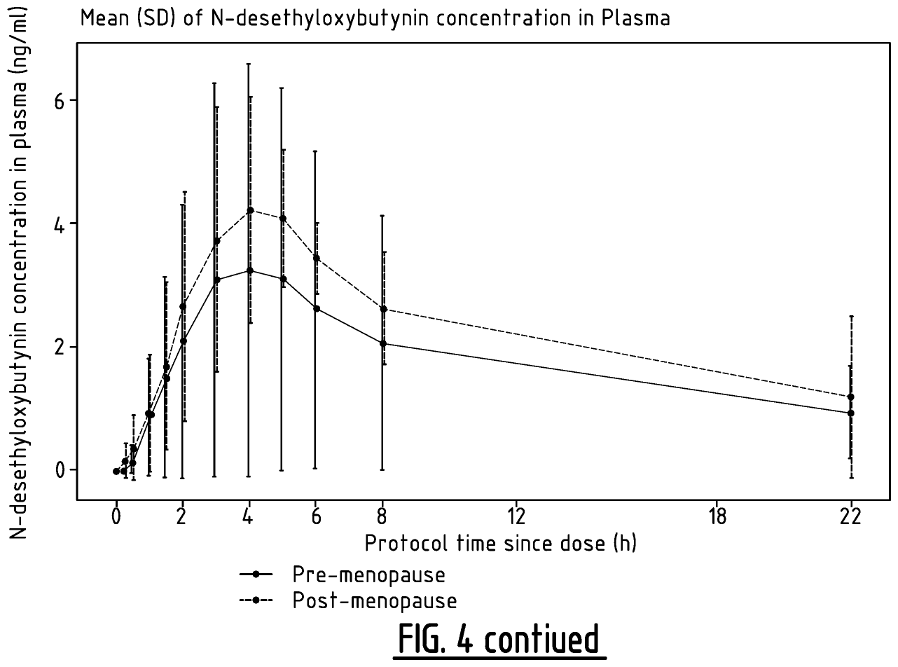

FIG. 4: Stratification of pharmacokinetic profiles by hormonal status. Oxybutynin (solid line: pre-menopausal subjects (n=4) and dashed line: postmenopausal subjects(n=4)) and N-desethyloxybutynin (solid line: pre-menopausal subjects (n=4) and dashed line: postmenopausal subjects(n=4)) concentration in plasma (ng/mL) are shown.

### EXAMPLE

### Administration of Oxybutynin to Subjects by Means of Vaginal Device

### INTRODUCTION

This trial investigated the feasibility of pulsed intra-vaginal delivery of oxybutynin and explored systemic exposure after application via the MedRing device.

### Materials and Methods

The trial included female subjects of childbearing potential (women of childbearing potential, WOCBP) aged between 18 and 45 years (inclusive) and female postmenopausal subjects aged between 50 and 69 years (inclusive); Postmenopausal status is defined as age≥50 years and having >12 months amenorrhoea in the absence of hormonal therapy that may cause amenorrhoea. The subjects were in good general health and had a body mass index between 18-32 kg·m² (inclusive) and with a minimum body weight of 50 kg at screening. Table 1 shows a summary of the subject participating in the trial.

TABLE 1

| Subject | Age | | | | Height | Weight | BMI |
|---|---|---|---|---|---|---|---|
| number | (years) | Gender | Race | Hormonal status | (cm) | (kg) | (kg/m²) |
| 1 | 33 | Female | Hispanic | Pre-menopausal | 162.5 | 59.95 | 22.7 |
| 2 | 31 | Female | White | Pre-menopausal | 170.6 | 67.25 | 23.0 |
| 3 | 21 | Female | Mixed | Pre-menopausal | 176.9 | 88.90 | 28.4 |
| | | | (White and | | | | |
| | | | Black) | | | | |
| 4 | 21 | Female | White | Pre-menopausal | 174.0 | 67.20 | 22.2 |
| 5 | 68 | Female | White | Post-menopausal | 168.6 | 87.30 | 30.7 |
| 6 | 69 | Female | White | Post-menopausal | 167.7 | 59.70 | 21.2 |
| 7 | 67 | Female | White | Post-menopausal | 165.2 | 69.15 | 25.3 |
| 8 | 57 | Female | White | Post-menopausal | 177.0 | 58.80 | 18.8 |

All subjects used contraceptives of second generation containing ethinylestradiol and progesterone derivate (WOCBP subjects only).

The MedRing was manufactured by Demcon Holding, The Netherlands, and filled with 100 mg/mL oxybutynin HCl. Oxybutynin HCl was dissolved in 50% propylene glycol/50% water for injection. The MedRing was pre-filled with 1-2 mL of oxybutynin 100 mg/mL, from which a controlled dose of up to 30 μL (=3 mg) was administered at least 15 minutes after intra-vaginal insertion of the MedRing by trained medical personnel.

Subjects began a 4-hour pre-dose fasting period before arriving to the clinic until 2 hours post-dose. Upon arrival, the subjects underwent pre-dose measurements to re-confirm eligibility for the study and to perform baseline measurements. The MedRing was inserted intra-vaginally by medically trained personnel. Water was allowed ad libitum. Subjects underwent blood sampling throughout the day. The MedRing was removed by medical study personnel 2 hours post dose in the first cohort. The MedRing was removed by medical personnel 6 hours post dose in the second cohort. The subjects went home approximately 8 hours post dose. The following day, the subjects of the second cohort returned to the clinic for an ambulant blood sample.

Subjects of the first cohort were dosed with 3 mg oxybutynin HCL with the MedRing that stayed in situ for 2 hours and subjects of the second cohort were dosed with 3 mg oxybutynin HCl with the MedRing that stayed in situ for 6 hours.

Plasma oxybutynin concentrations were measured by a validated LC/MS/MS method by Ardena Bioanalytical Laboratories, Assen, The Netherlands. Plasma samples were also used for assessment of the concentrations of the metabolite, N-desethyloxybutynin.

### Results

Oxybutynin was absorbed by all eight (8) subjects with no apparent differences in absorption, distribution and elimination between subjects that could be explained by pre- and postmenopausal status or from keeping the MedRing in situ for 2 or 6 hours, as can be observed in FIG. 2 (individual plasma concentration graphs), FIG. 3 (in situ time MedRing) and FIG. 4 (hormonal status).

At 3 mg oxybutynin HCL administered intravaginally, plasma oxybutynin concentrations peaked rapidly (median: 2 hours) after administration and declined with a mean t1/2 of 8.5 hours. As expected, plasma concentrations of the major metabolite N-desethyloxybutynin peaked slightly later (median: 4 hours) and declined over a longer period with a mean t1/2 of 7.7 hours.

The observed mean (±standard deviation) Cmax of oxybutynin in plasma was 5.4 ng/ml (±2.7), observed at a median (min-max) tmax 2 (1-5) hours post dose with an estimated mean (±SD) t1/2 of 8.5 (±3.5) hours. The Cmax of the metabolite, N-desethyloxybutynin, reached 3.9 ng/ml (+2.5) in plasma, observed at a median (min-max) Tmax 4 (3-5) hours post dose with a mean (±SD) t1/2 of 7.7 hours (±5.9). After 22 hours, both oxybutynin and N-desethyloxybutynin levels were found in all 4 subjects (no 22-hour sample was obtained in the other 4 subjects).

There were no major differences in pharmacokinetic parameters between the two cohorts or subjects based on hormonal status (pre- or postmenopausal).

The observed mean (±SD) Area under the concentration—time curve to infinity (AUCinf) of oxybutynin and N-desethyloxybutynin in plasma was 34.9 (±17.4) and 51.1 (±43.1), respectively. The parent:metabolite ratio on the AUCinf was calculated for individuals who had successful linear regression of apparent elimination phases of both parent and metabolite concentrations. This resulted in a parent:metabolite ratio of 1:1.3.

The parent:metabolite ratio for oral immediate release formulations of oxybutynin is 1:5.5, whilst of an oral extended release this ratio is 1:4.3. Transdermal patch formulation results in a parent:metabolite ratio of 1:1.3 and the transdermal gel to a ratio of 1:0.8. It follows that the vaginal administration leads to a reduced level of the metabolite as a result of the absence of the hepatic first past effect in vaginal administration, while avoiding the side effects of transdermal applications.

1. Oxybutynin for use in the treatment of a medical condition, wherein oxybutynin is administered to the subject in need of treatment by means of a vaginal ring, that comprises a drug reservoir for the liquid formulation of oxybutynin and a pump for dispensing the oxybutynin.

2. Oxybutynin as claimed in claim 1, wherein the medical condition comprises overactive bladder (OAB).

3. Oxybutynin as claimed in claim 2, wherein the oxybutynin is administered via the ring in a range of 1-5, preferably about 3 mg in liquid formulation.

4. Oxybutynin as claimed in claim 1, wherein the medical condition comprises treatment of post-menopausal or aromatase inhibitor-induced hot flashes.

5. Oxybutynin as claimed in any one of the claims 1-4, wherein the pump of the vaginal ring comprises a miniature peristaltic pump.

6. Oxybutynin as claimed in claim any one of the claims 1-5, wherein the vaginal ring further comprises a miniature electronic circuit board that controls the ring, and optionally a battery.

7. Oxybutynin as claimed in any one of claims 1-6, wherein the vaginal ring is wirelessly connected to an external device for programming drug delivery.

8. Oxybutynin as claimed in claim 5 or 6, wherein the vaginal ring is wirelessly connected to an external device for transmitting and/or receiving data from the ring.

9. Oxybutynin as claimed in any one of the claims 5-8, wherein the external device is a smartphone, tablet, or laptop computer.

10. Oxybutynin as claimed in any one of the claims 5-9, wherein the data received by the external device comprises volume delivered, temperature.

11. Oxybutynin as claimed in any one of the claims 5-10, wherein the data transmitted by the external device comprises volume to be delivered, point of time of delivery, delivery pattern.
